(19) United States
(12) Patent Application Publication
(10) Pub. No.: US 2023/0095595 A1
(43) Pub. Date: Mar. 30, 2023

(54) METHOD AND SYSTEM FOR DISPLAYING A PREDETERMINED SEQUENCE OF EXERCISE ROUTINE

(71) Applicants: Lynette Deschler, Lake Oswega, OR (US); John Kasha, Simi Valley, CA (US)

(72) Inventors: Lynette Deschler, Lake Oswega, OR (US); John Kasha, Simi Valley, CA (US)

(73) Assignee: TH2MAT, LLC, Lake Oswego, OR (US)

(21) Appl. No.: 17/448,912

(22) Filed: Sep. 27, 2021

(51) Int. Cl. A63B 24/00; A63B 21/00; A63B 71/06

(52) U.S. Cl. CPC A63B 24/0075; A63B 21/4037; A63B 24/0062; A63B 71/0622; A63B 2024/0078; A63B 2071/0694; A63B 2225/50; A63B 2220/62; A63B 2225/74

(57) ABSTRACT

A computer-implemented method for displaying a predetermined sequence of exercise routines is provided. The method includes receiving a light sequence for the predetermined sequence of exercise routine from an application interface of a user device. The method further includes triggering one or more light emitting elements of a set of light emitting elements disposed on an exercising mat and detecting a movement on one or more touch buttons of a set of touch buttons on the exercising mat. The method includes determining a state of each of the corresponding one or more touch buttons upon detection of the movement. Furthermore, the method includes activating a debounce mechanism to delay in illuminating next one or more light emitting elements of the set of light emitting elements based on the state and displaying the light sequence on the exercising mat for guiding a user for performing the predetermined sequence of exercise routines.

### TECHNOLOGICAL FIELD

The present disclosure generally relates to an exercise system, and more particularly relates to a method and a system for displaying a predetermined sequence of exercise routine.

### BACKGROUND

When a human body moves to perform daily activities or exercises, the body moves in different dimensions. The human body makes movements, such as forward and backward, side-to-side, up, and down and rotation occurring in different planes of movements. The different planes of movements (also known as anatomical planes) include a frontal plane, a sagittal plane, and a transverse plane. The frontal plane divides the body into front and back, the sagittal plane divides the body into right and left sides and the transverse plane divides the body into top and bottom sections. However, traditional workout and strength training routines often involve movements in a single plane of movement. For instance, a biceps curl, a forward or reverse lunge, a squat, vertical jumping, running, a downward dog, or chair pose (in yoga), occur in the sagittal plane. These workout plans and strength training routines are often repetitive and emphasize only on physical strength, without much mental involvement. For example, when a person performs a push-up exercise, upper body of the person is pushed up and down repeatedly in the transverse plane with little engagement of the mind. The repeated movement with the lack of mental involvement may prevent the body from achieving an optimal physical strength. This reduces the benefit of the exercise. For instance, strength of connective tissues of both mind and body may reduce due to the repeated movement, while causing wear and tear to the connective tissues.

Accordingly, there is a need for a solution to overcome the above-mentioned limitation. More specifically, there is a need to for a method and a system for displaying a predetermined sequence of exercise routine to perform exercises in an efficient manner.

### BRIEF SUMMARY

In order to solve the foregoing problem, the present disclosure provides a system and a method for displaying a predetermined sequence of exercise routine for performing exercise movements in an efficient manner. In some embodiments, the predetermined sequence of exercise routine is displayed through a light sequence of one or more light emitting elements on an exercising mat. The light sequence guides a user to perform the predetermined sequence of exercise routine occurring in one or more anatomical planes of a 3D human movement. The anatomical planes include a frontal plane, a sagittal plane, and a transverse plane. The frontal plane corresponds to side-to-side movements, the sagittal plane corresponds to forward and backward movements, and the transverse plane corresponds to rotation movements.

The one or more light emitting elements correspond to a set of light emitting elements disposed on a top surface of the exercising mat. The set of light emitting elements is arranged according to the anatomical planes. The set of light emitting elements is also operably coupled to a set of touch buttons embedded within the exercising mat. The set of touch buttons detect movements of the user corresponding to the predetermined sequence of exercise routine. When the user steps on the one or more light emitting elements, corresponding one or more touch buttons connected with the one or more light emitting elements detect the movements. The detected movements switch on the one or more touch buttons that illuminate consequent one or more light emitting elements for guiding consequent movements of the predetermined sequence of exercise routine. The set of light emitting elements and the set of touch buttons are connected to an electronic circuitry integrated to the exercising mat.

The electronic circuitry establishes a wireless connection with an application interface of a user device for receiving the light sequence. When the wireless connection is established, the set of light emitting elements and the set of touch buttons are synchronized with a corresponding set of virtual light emitting elements and a set of virtual touch buttons in the application interface. After the synchronization, a test program is executed to test the set of light emitting elements. The set of light emitting elements are turned on based on the executed test program. Further, a list of predetermined sequence of exercise routines is displayed in the application interface. The user selects a predetermined sequence of exercise routine from the list of predetermined sequence of exercise routines. Furthermore, a list of game options for the selected predetermined sequence of exercise routine is displayed in the application interface. The list of game options may include one or more of a mental and physical coordination game option, a memory game option, a physical strength game option, a speed movement game option and/or the like. The user selects a game option for the predetermined sequence of exercise routine from the list of game options. In some cases, the user may view a demonstration for the predetermined sequence of exercise routine of the particular game option from the application interface. After the user selection, the application interface sends the light sequence for the selected predetermined sequence of exercise routine to the electronic circuitry of the exercising mat.

The one or more light emitting elements on the exercising mat are illuminated based on the light sequence. When the user steps on the illuminated light emitting elements, touch buttons connected with the corresponding light emitting elements detect movement on the user. When the movements are detected, a state of each of the corresponding one or more touch buttons is determined. The determined state is used to activate a debounce mechanism to introduce a delay in illuminating next one or more light emitting elements of the set of light emitting elements. The delay in illuminating the next or more light emitting elements may be achieved by preventing spike in signal of the one or more touch buttons based on the debounce mechanism. The debounce mechanism may be implemented via a firmware of the electronic circuitry. The debounce mechanism includes triggering a debounce wait time when a value of the determined state is true. That is, the debounce wait time is triggered and the state is set true, when a movement is detected on corresponding touch button. In case, the value of the determined state is false, the debounce wait time is cancelled and the delay is deactivated. Such debounce mechanism on the exercising mat prevents signal spike through ground, which in turn avoids changes in operation of the electronic circuitry.

When the user starts performing the movement on the exercising mat, the detected movement is shared to the application interface by the electronic circuitry. The application interface then notes time duration that the user started the movement. The time duration is stopped when the application interface receives a user input for marking a completion of the predetermined sequence of exercise routine. After the completion of the predetermined sequence of exercise routine, the application interface displays a total time duration taken to complete the predetermined sequence of exercise routine and a number of rounds of the predetermined sequence of exercise routine performed by the user. The application interface also displays a statistical report indicative of a progress rating of the user corresponding to the completion of the predetermined sequence of exercise routine.

In this way, the system provided herein enables the user to perform movements of the predetermined sequence of exercise routine occurring in one or more anatomical planes. The movements in one or more anatomical planes may maximize exercise performance, while reducing risk of injury.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

Having thus described example embodiments of the invention in general terms, reference will now be made to the accompanying drawings, which are not necessarily drawn to scale, and wherein:

FIG. 1 illustrates an environment representation of a system for displaying a predetermined sequence of exercise routine, in accordance with one or more example embodiments.

FIG. 2 illustrates a sequence of graphical user interfaces (GUIs) of an application interface of a user device, in accordance with one or more example embodiments.

FIG. 3 illustrates a diagrammatic representation of an exercising mat, in accordance with one or more example embodiments.

FIG. 4 illustrates a schematic block diagram of the exercising mat of FIG. 3, in accordance with one or more example embodiments.

FIG. 5A illustrates a block diagram of an electronic circuitry of the exercising mat, in accordance with one or more example embodiments.

FIG. 5B illustrates a detailed block diagram of an electronic circuitry of the exercising mat, in accordance with one or more example embodiments.

FIGS. 6A and 6B show a flowchart illustrating a process for displaying a predetermined sequence of exercise routine, in accordance with one or more example embodiments.

FIG. 7 shows a flowchart illustrating a process for initializing the electronic circuitry of the exercising mat, in accordance with one or more example embodiments.

FIG. 8 illustrates different states in a debounce mechanism corresponding to display of the predetermined sequence of exercise routine, in accordance with one or more example embodiments.

FIGS. 9A and 9B show a flowchart illustrating a process corresponding to interfacing of the application interface by the user, in accordance with one or more example embodiments.

FIGS. 10A and 10B shows a flowchart illustrating a process for displaying a list of predetermined sequence of exercise routines and a list of game options, in accordance with one or more example embodiments.

FIG. 11 shows a flowchart illustrating a process corresponding to a selected predetermined sequence of exercise of a selected game option, in accordance with one or more example embodiments.

FIG. 12 shows a flowchart illustrating a process corresponding to a selected predetermined sequence of exercise routine of a selected game option, in accordance with one or more example embodiments.

FIG. 13 shows another flowchart illustrating a process corresponding to a selected predetermined sequence of exercise routine of a selected game option, in accordance with one or more example embodiments.

FIG. 14 shows a block diagram of a server hosting the application interface for displaying the predetermined sequence of exercise routine, in accordance with one or more example embodiments.

FIGS. 15A, 15B and 15C illustrates an example use case for displaying a predetermined sequence of exercise routine, in accordance with one or more example embodiments.

FIG. 16 illustrates another example use case for displaying a predetermined sequence of exercise routine, in accordance with one or more example embodiments.

FIG. 17 shows a method flow for displaying a predetermined sequence of exercise routine, in accordance with one or more example embodiments.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, apparatuses and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Additionally, as used herein, the term ‘circuitry’ may refer to (a) hardware-only circuit implementations (for example, implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

As defined herein, a “computer-readable storage medium,” which refers to a non-transitory physical storage medium (for example, volatile or non-volatile memory device), may be differentiated from a “computer-readable transmission medium,” which refers to an electromagnetic signal.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

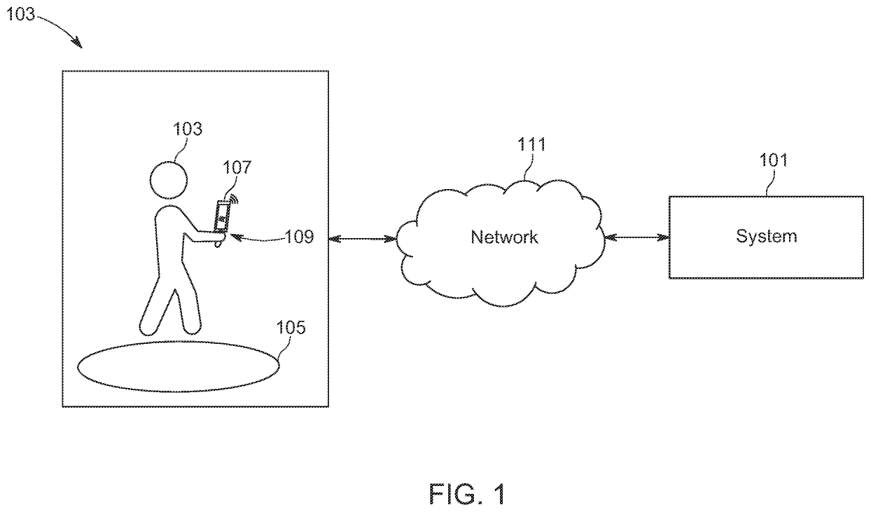

FIG. 1 illustrates an environment representation 100 of a system for displaying a predetermined sequence of exercise routine to a user 103, in accordance with one or more example embodiments. In some embodiments, the predetermined sequence of exercise routine is displayed on an exercising mat 105 using an application interface 109 in a user device 107. The user device 107 includes one or more processors, a memory, and I/O interface. The user device 107 corresponds to a smart phone, a laptop, a computer, a tablet, a smart watch, and the like.

The application interface 109 may be hosted by a system 101. The user device 107 may communicate with the system 101 using the application interface 109 via a network 111. The network 111 may be wired, wireless, or any combination of wired and wireless communication networks, such as cellular, Wi-Fi, internet, local area networks, or the like. The system 101 includes a server (for instance, a backend server, a remotely located server, or the like), group of servers, distributed computing system, and/or other computing system.

The system 101 may be configured to generate a predetermined sequence of exercise routine for each of a plurality of exercises for different users, such as the user 103. The predetermined sequence of exercise routine for each of the plurality of exercises, information of the plurality of exercises, and information of the different users may be stored and maintained in a database (not shown) associated with the system 101. The system 101 may also be configured to generate different types of game options for each of the plurality of the exercises. The plurality of exercises may each have an associated predetermined sequence of exercise routine, which is displayed through a light sequence on the exercising mat 105. Such light sequence may be stored in the database associated with the system 101. The information about the light sequence may transmitted by the user device 101 via the application interface 109 to the exercising mat 105 upon receiving a user selection for the corresponding predetermined sequence of exercise routine on the application interface 109. The exercising mat 105 includes an electronic circuitry that may be configured to establish a wireless connection with the application interface 109 and receive the transmitted light sequence from the application interface 109 via the wireless connection. For instance, the exercising mat 105 and the application interface 109 may be connected via the electronic circuitry using the network 111 for enabling the wireless connection.

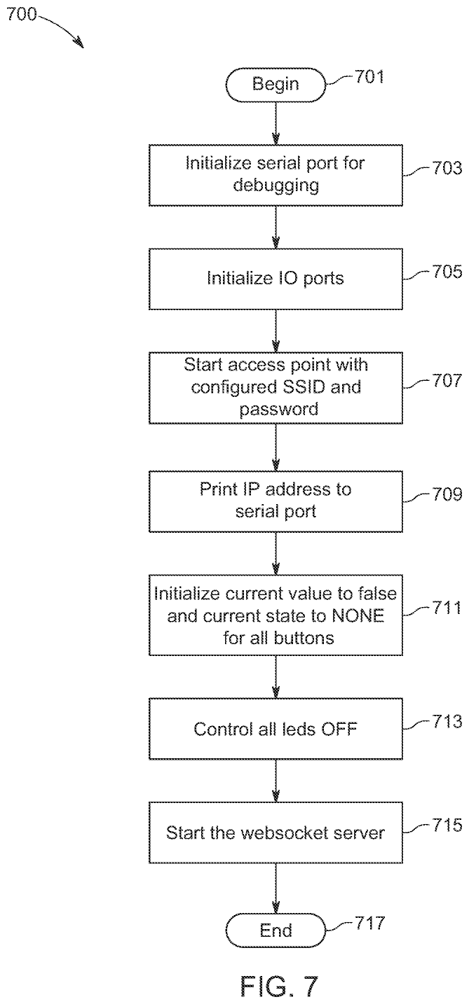

In some example embodiments, the application interface 109 may receive a user input for establishing the wireless connection. The user input may include a connection identifier, such as an Internet Protocol (IP) address for the wireless connection. When the wireless connection is established, synchronization may be performed between the application interface 109 and the exercising mat 105 via the electronic circuitry. The synchronization corresponds to synchronizing a set of light emitting elements of the exercising mat 105 and a set of touch buttons coupled with the set of light emitting elements with a set of virtual light emitting elements and a set of virtual touch buttons in the application interface 109. The set of light emitting elements may be disposed on a top surface of the exercising mat 105. The set of touch buttons may be embedded within the exercising mat 105. After the synchronization, the application interface 109 may execute a test for the set of light emitting elements. When the test is successful, the application interface 109 may continue displaying a list of predetermined sequence of exercise routines and a list of game options to the user 103. In case the test fails, then the electronic circuitry is debugged, which will be described later in conjunction with FIG. 7.

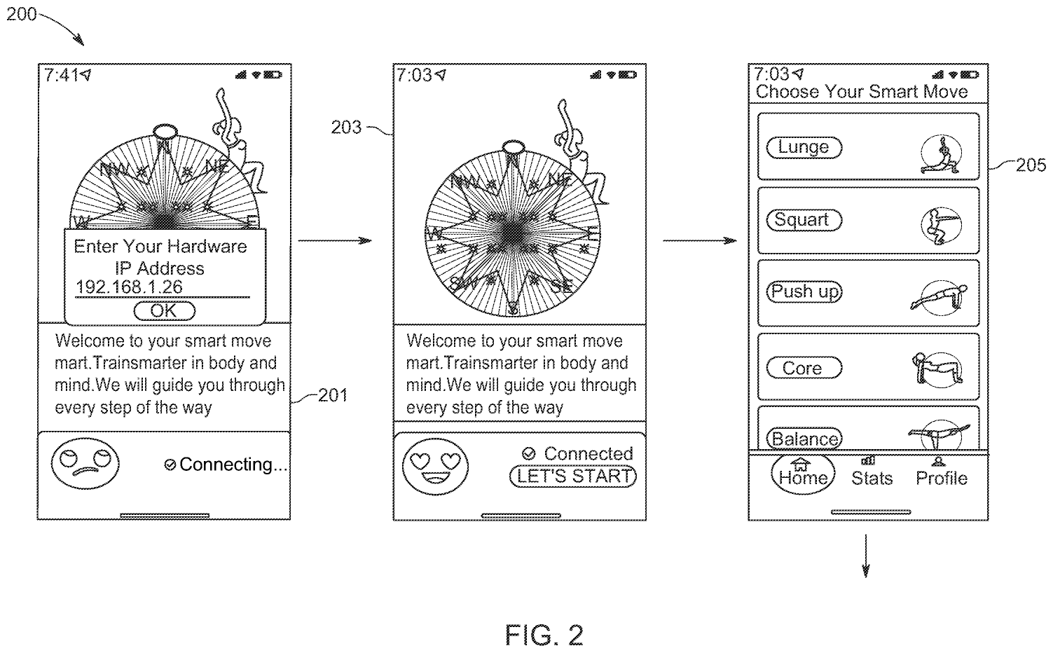

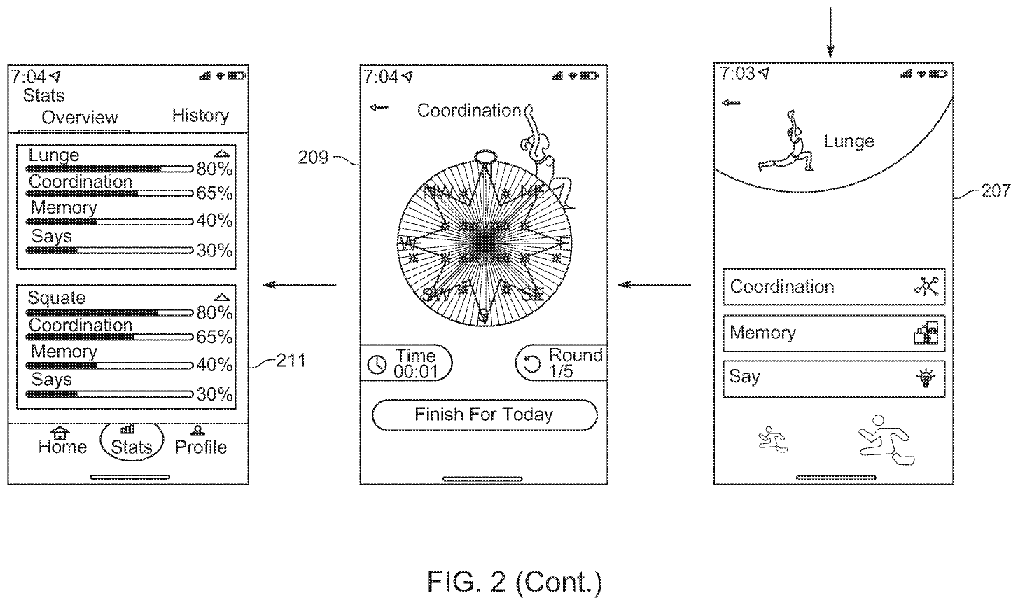

The establishment of the wireless connection, the synchronization and the display of the exercise routines and the game options, are further explained in the detailed description of FIG. 2.

FIG. 2 shows a sequence 200 of graphical user interfaces (GUIs), such as a GUI 201, a GUI 203, a GUI 205, a GUI 207, a GUI 209, and a GUI 211 of the application interface 109, in accordance with one or more example embodiments. When the application interface 109 is started, a welcome message is displayed to the user 103, as shown in GUI 201 of FIG. 2. The GUI 201 also includes an input field for a connection identifier and a button, such as an “OK” button. The connection identifier may correspond to an IP address for wirelessly connecting to an electronic circuitry of the exercising mat 105, via a network, such as the network 111 of FIG. 1. The user 103 may click on the OK button to establish the wireless connection upon filling the IP address in the input field.

After the wireless connection is established, a set of virtual light emitting elements and a status of the wireless connection are displayed in the GUI 203. The set of virtual light emitting elements is a visual representation of the set of light emitting elements of the exercising mat 105. The GUI 203 also includes a start button, such as “LET'S START” button. The user 103 may click on the start button to start an exercise routine. In the GUI 205, a list of predetermined sequence of exercise routines corresponding to different types of movements or exercises is displayed. For instance, the list of predetermined sequence of exercise routines includes a lunge exercise, a squat exercise, a push-up exercise, a core exercise, a balance exercise, or the like. The user 103 may select one option, for example, the lunge exercise from the list of predetermined sequence of exercise routines. For each of these exercises, there may be a variety of game options or game options. As shown in GUI 207, for the lunge exercise, there are three options, such as “coordination” option, “memory” option and “say” option. The user 103 may select the “coordination” option from the three options. In the GUI 209, a light sequence corresponding to a sequence of movement for the lunge exercise is shown. The light sequence is shared to the exercising mat 105 from the application interface 109. The light sequence is illuminated through one or more light emitting elements on the exercising mat 105 that guides the user 103 to perform the lunge exercise on the exercising mat 105. After performing the exercise, the user 103 may click on a finish button, such as “FINISH FOR TODAY” button, as shown in GUI 209. In the GUI 211, a statistical report indicative of a progress rating of the user 103 corresponding to completion of the lunge exercise is displayed.

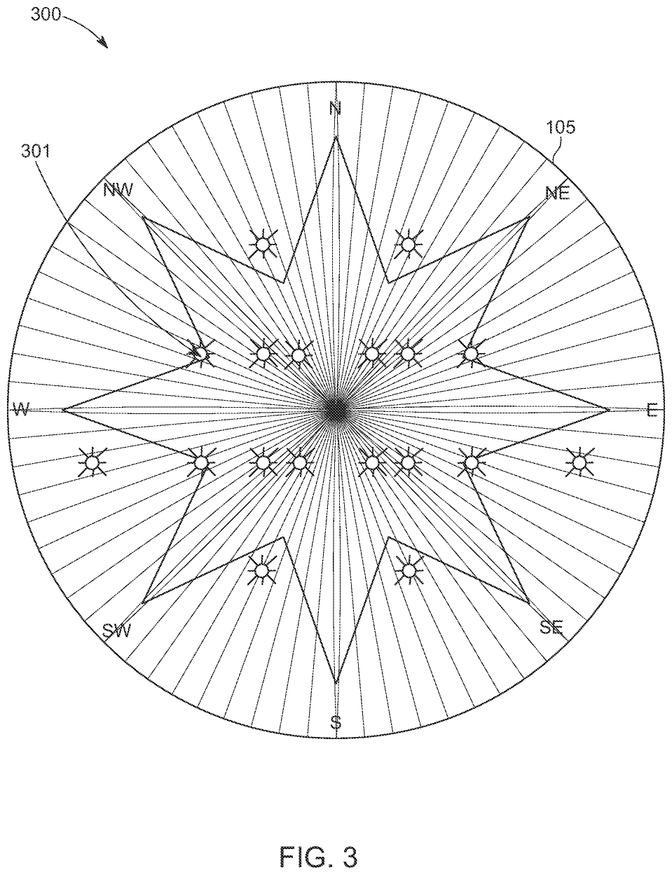

The exercising mat 105 includes a set of light emitting elements for guiding the user 103 for performing the predetermined sequence of exercise routine. The exercising mat 105 with the set of light emitting elements is described next in FIG. 3.

FIG. 3 illustrates a diagrammatic representation 300 of the exercising mat 105, in accordance with one or more example embodiments. In some example embodiments, the exercising mat 105 is in circular shape and layered with lightweight materials. For instance, the exercising mat 105 may have the circular shape of 6 feet (1.82 meters) in diameter and the lightweight materials may include a light canvas, layered thin transparent plastic sheets, conductive paint coats, foam lining, graphics designs, or the like. In some embodiments, a set of light emitting elements collectively referred to hereinafter as a set of light emitting elements 301 is disposed on a top surface of the exercising mat 105. As shown in FIG. 3, the set of light emitting elements is indicated by white dots on the exercising mat 105. The set of light emitting elements corresponds to light emitting diodes (LEDs) that is disposed together with a corresponding set of touch buttons (not shown). The set of touch buttons may be embedded within the exercising mat 105. In some embodiments, the set of the touch buttons corresponds to momentary switch buttons, or pressure switch buttons, such as mechanical switch buttons, electronic switch buttons, or the like. The pressure switch buttons may be triggered when the user 103 steps on corresponding illuminated light emitting elements on the exercising mat 105.

In some embodiments, the set of light emitting elements and the set of touch buttons are arranged corresponding to one or more anatomical planes of a human body, i.e., a frontal plane, a sagittal plane, and a transverse plane. The arrangement of the set of light emitting elements and the set of touch buttons in the anatomical planes may maximize movements of the user 103, while performing the predetermined sequence of exercise routine (such as the sequence of exercise routine for the lunge exercise) on the exercising mat.

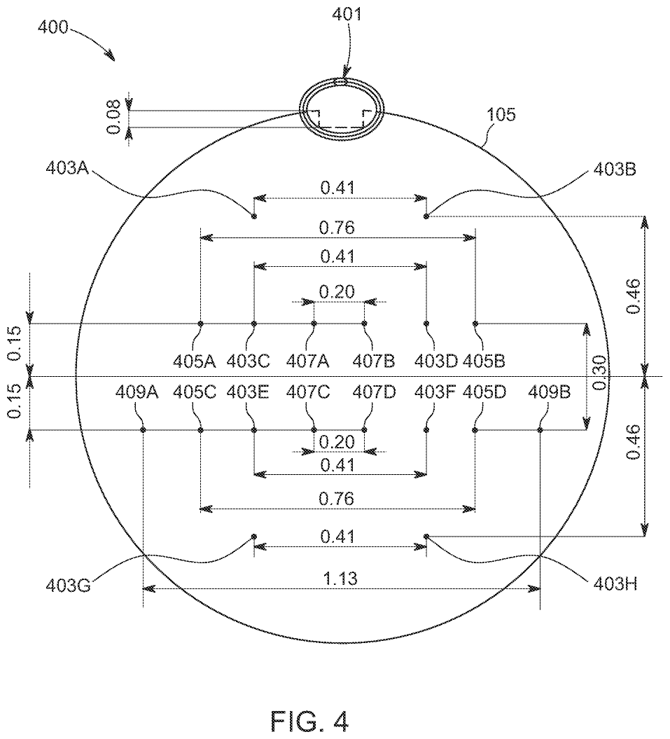

The arrangement of the set of light emitting elements and the set of touch buttons is further explained in FIG. 4.

FIG. 4 illustrates a schematic block diagram 400 of the exercising mat 105, in accordance with one or more example embodiments. In some embodiments, the exercising mat 105 is mounted with an electronic circuitry 401. The electronic circuitry 401 is operably connected to the set of light emitting elements disposed on top of the exercise mat 105 and the set of touch buttons disposed inside the exercise mat 105. The set of light set of light emitting elements and the set of touch buttons are disposed at locations indicated by points, such as points 403A, 403B, 403C, 403D, 403E, 403F, 403G and 403H, points 405A, 405B, 405C and 405D, points 407A, 407B, 407C and 407D, and points 409A and 409B. As shown in FIG. 4, the light emitting elements and the set of touch buttons located at the points 403A, 403C, 403E, and 403G are 0.41 feet apart from the light emitting elements and the set of touch buttons located at the corresponding points 403B, 403D, 403F, and 403H. The light emitting elements and the set of touch buttons at the points 405A and 405C are 0.76 feet apart from the light emitting elements and the touch buttons located at the corresponding points 405B and 405D. The light emitting elements and the touch buttons at the points 407A and 407C are 0.20 feet apart from the light emitting elements and the touch buttons located at the corresponding points 407B, and 407D. The light emitting element and the touch button at the point 409A are 1.31 feet apart from the light emitting element and the touch button at the point 409B.

The light emitting elements and the touch buttons at the points 403A and 403B are 0.46 feet away from a horizontal mid-line of the exercising mat 105. In a similar manner, the light emitting elements and the touch buttons at the points 403G and 403H are 0.46 feet away from the horizontal mid-line of the exercising mat 105. The light emitting elements and the touch buttons at the points 403C, 403D, 405A, 405B, 407A, and 407B are 0.15 feet away from the horizontal mid-line of the exercising mat 105. Likewise, the light emitting elements and the touch buttons at the points 403E, 403F, 405C, 405D, 407A, 407B, 409A and 409B are 0.15 feet away from the horizontal mid-line of the exercising mat 105. It may be understood by a person of ordinary skill in the art that the set of distances between the various points shown in the FIG. 4 are for exemplary purpose only. Any other equivalent distance values may be implemented for carrying out the functions of the disclosure, without deviating from the scope of the present disclosure.

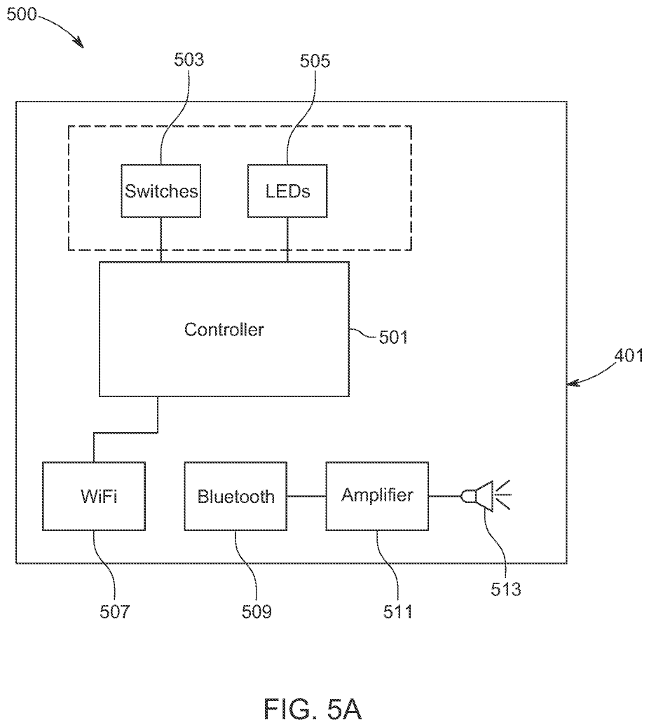

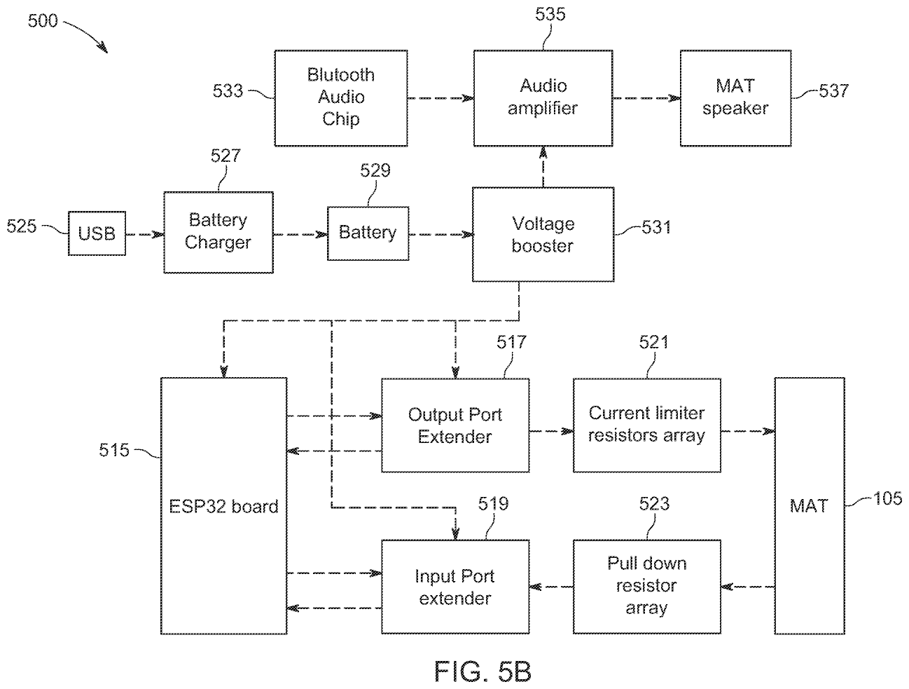

As mentioned earlier, the light emitting elements are illuminated based on the light sequence received by the electronic circuitry 401 from the application interface 109. When the user 103 steps on the illuminated light emitting elements, the corresponding touch buttons are triggered to turn on. The triggered touch buttons enable the electronic circuitry 401 to detect movement of the user 103 on the exercising mat 105. The detection of the movement by the electronic circuitry 401 from the touch buttons is further explained with reference to FIG. 5.

FIG. 5A illustrates a block diagram 500A of the electronic circuitry 401 of the exercising mat 105, in accordance with one or more example embodiments. The electronic circuitry 401 includes a controller 501 for controlling the set of light emitting elements 301, such as LEDs 505 and a set of touch buttons, such as switches 503. The controller 501 includes a series of Input/Output (IO) ports for connecting each of the LEDs 505 and the switches 503.

The electronic circuitry 401 further includes a Wi-Fi module 507, a Bluetooth module 509, an amplifier 511 and an audio output, such as a speaker 513. The Wi-Fi 33 module 507 may enable the controller 501 ability to access to a wireless network, such as the network 111. This enables the electronic circuitry 401 to connect to the application interface 109 using the Wi-Fi module 507. Some of the non-limiting examples of the Wi-Fi module 507 include ESP8266 Wi-Fi Bee, ESP32 Wi-Fi module, WT8266-S1 Wi-Fi module, or the like. The Bluetooth module 509 (for example, HC-05 Bluetooth Module) may include a circuit set of a chip with integrated Bluetooth function for short-distance wireless audio transmission. The Bluetooth module 509 may be connected to the amplifier 511 and the speaker 513. The amplifier 511 may amplify an audio that is outputted via the speaker 513. The audio may include audio commands, instructions, audio music, or the like corresponding to the predetermined sequence of exercise routine.

In some embodiments, the controller 501 may be embedded with a firmware that includes instructions to synchronize the switches 503 and the LEDs 505 with corresponding virtual set of light emitting elements and virtual set of touch buttons in the application interface 109. The firmware may also include instructions to sense a switch closure of the switches 503. For instance, when the user 103 steps on an illuminated light emitting element, a touch button connected with the illuminated light emitting element is triggered, which results in the switch closure. The switch closure turns on the touch button that is sensed by the controller 501 based on the firmware. When the user 103 moves away from the illuminated light emitting element, there is no occurrence of switch closure resulting in deactivation of the touch button. That is, the touch button is turned off when the user 103 steps away due to lack of the switch closure. In this manner, the controller 501 senses the movement of the user 103 based on activation and deactivation of the switch closure of each of the touch buttons, i.e., the switches 503.

In some cases, the user 103 may walk over the exercising mat 105. The user 103 may randomly step on one or more of touch buttons, while walking on the exercising mat 105. In such cases, one or more of the switches 503 may undergo switch closures and the one or more of the switches 503 may be triggered to turn on. The trigger of more than one of the switches 503 may spike multiple signals and spike through ground. The spike in signal may cause the controller 501 to generate false logic changes, resulting in wrong illumination of the LEDs 505. To that end, the firmware may further include instructions to debounce or create a slight delay in triggering the switches 503 to prevent the spike due to the random switch closures. Such debounce mechanism enables the controller 501 to allow only one signal to act upon a switch closure of one of the switches 503. Additionally, or alternatively, the exercising mat 105 may be coated with a conductive paint that offers a slight resistance of about 1 K ohms to overcome the spike through the ground. The debounce mechanism may be optional if the switches 503 correspond to mechanical switch buttons. In case the switches 503 correspond to electronic switch buttons, there may be signal spike due to the random closure and the debounce mechanism may be mandatory.

In some example embodiments, the electronic circuitry 401 may further electronic components for implementing the debounce mechanism, which is further described with reference to FIG. 5B.

FIG. 5B illustrates a detailed block diagram 500B of the electronic circuitry 401 of the exercising mat 105, in accordance with one or more example embodiments. The electronic circuitry 401 may also include a printer circuit board (PCB) 515, such as an ESP32 board. The ESP32 board is low-power system on a chip microcontroller that is connected as a slave device to a host microcontroller, such as the controller 501. The ESP32 board 515 may reducing communication stack overhead on the controller 501. The PCB 515 is further connected to an output port extender 517 and an input port extender 519. The output port extender 517 and the input port extender 519 monitor and control peripheral signals of components of the electronic circuitry 401.

The output port extender 517 is connected to a current limiting resistors array 521. The current limiting resistor array 521 is a resistor that is used to control current in the electronic circuitry 401. The current limiting resistor 521 may be resistor connected in series with the LEDs 505. For instance, the current limiter resistors array 521 controls amount of current through the LEDs 505.

The input port extender 519 is connected to a pull-down resistors array 523. The pull-down resistors array 523 control a microcontroller pin of the PCB 515 to a low value. For instance, the pull-down resistors array 523 may connect unused input pins (e.g., OR and NOR gates) of the PCB 515 to ground, (0V) to keep the input value low. The PCB 515 is further connected to a voltage booster 531 to boost a voltage supply from a battery 529 to the controller 501. The battery 529 is connected to a battery charger 527 that stores charge for operating the controller 501. The charge may be from a power supply that is connected via a universal serial bus (USB). The power is also supplied to a Bluetooth audio chip 533 via the voltage booster 531. The Bluetooth audio chip 533 is connected to an audio amplifier 535 and a mat speaker 537. The Bluetooth audio chip 533 corresponds to the Bluetooth module 509, the audio amplifier 535 corresponds to the amplifier 511 and the mat speaker 537 corresponds to the speaker 513.

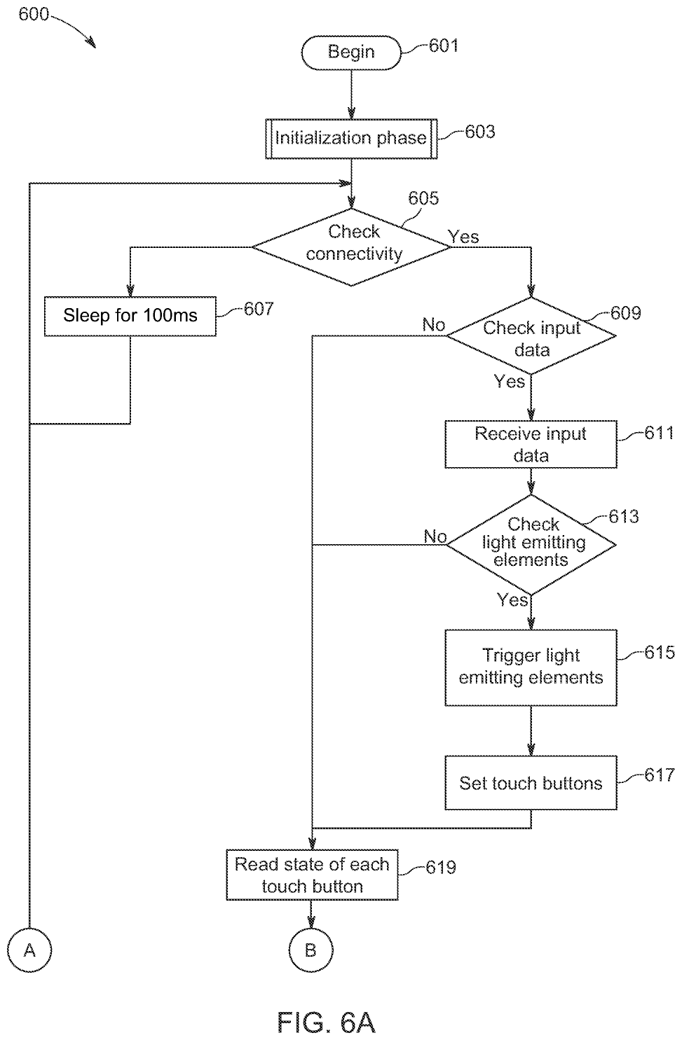

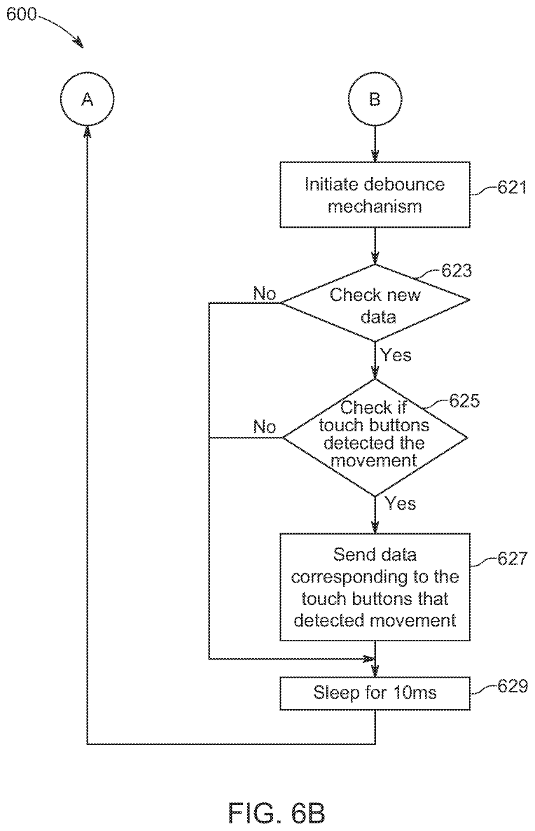

The electronic circuitry shown by virtue of block diagrams of FIGS. 5A and 5B are configured to implement displaying of a predetermined sequence of exercise routine on the exercise mat 105, as will be further explained in FIG. 6A and FIG. 6B described below.

The steps for displaying a predetermined sequence of exercise routine are described in FIGS. 6A and 6B.

FIGS. 6A and 6B show a flowchart illustrating a process 600 for displaying a predetermined sequence of exercise routine, in accordance with one or more example embodiments. The process 600 is executed by the controller 501. At step 601, the process 600 begins. At step 603, the electronic circuitry 401 is initialized. The electronic circuitry 401 is initialized when a wireless connection is established between the electronic circuitry 401 of the exercising mat 105 and the application interface 109 in the user device 107. The initialization of the electronic circuitry 105 is further described in detail in FIG. 7.

At step 605, the controller 501 checks if there is connectivity of the exercise mat 105 with the application interface 109. At step 607, if it is determined that there is no connectivity yet between the exercise mat 105 and the application interface 109, then the controller 501 is configured to cause the processing to get deactivated and this is referred to as the controller 501 “sleeps” for a predefined time-period, such as 100 milliseconds (ms). However, if at 605 it is determined that there is connectivity between the exercise mat 105 and the application interface 109, then at step 609, the controller 501 checks for an input data from the application interface 109. The input data may correspond to a light sequence for guiding a predetermined sequence of exercise routine on the exercising mat 105. The light sequence includes information of one or more light emitting elements of the set of light emitting elements 301 on the exercise mat 105. In case, there is no input data detected from the application interface 109, then the controller 501 proceeds to step 619.

In case, if at 609 the input data is detected, then at step 611, the controller 501 receives the input data that includes the light sequence for guiding the predetermined sequence of exercise routine on the exercising mat 105. At step 613, the information of the one or more light emitting elements in the light sequence is checked by the controller 501. The controller 501 may detect an error, a flaw, or a fault in the information of the one or more light emitting elements that may cause an incorrect display of the light sequence for the predetermined sequence of exercise routine on the exercising mat 105. For instance, the information of the one or more light emitting elements may not be correctly or adequately received at step 613. If the error is detected, then the control passes to step 619. If there is no detection of the error in the information of the one or more light emitting elements at step 613, then at step 615, the one or more light emitting elements in the light sequence are triggered. For instance, the controller 501 sends a request or a command to trigger the one or more light emitting elements on the exercising mat 105.

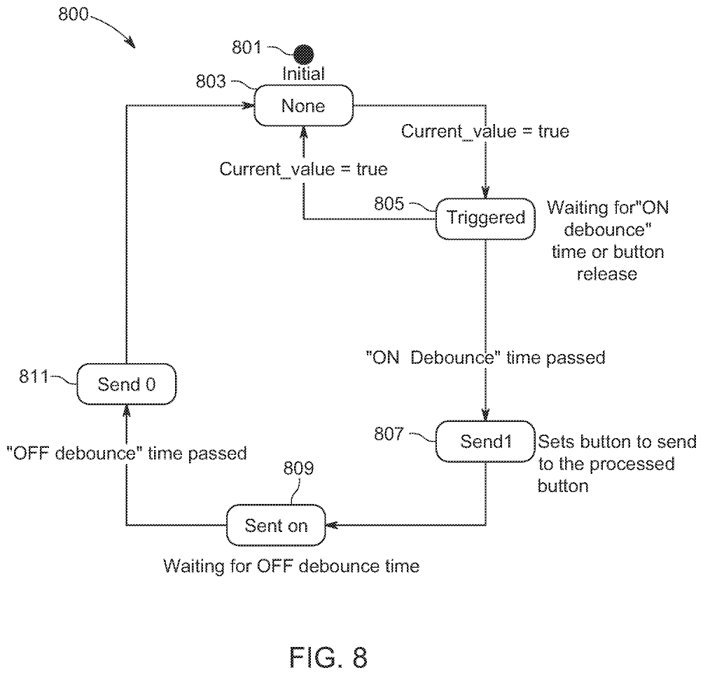

Next, at step 617, one or more touch buttons of the set of touch buttons on the exercise mat 105 are set corresponding to the requested one or more light emitting elements. For instance, the one or more touch buttons are activated based on the triggered one or more light emitting elements. Further, the control passes to step 619. At step 619, d of each of the one or more touch buttons are read. Based on this reading, at step 621, a debounce mechanism is initiated. The debounce mechanism creates a slight delay in setting the corresponding one or more touch buttons so that a spike in signal of the one or more touch buttons do not cause spikes through ground and prevent generating false logic changes in the electronic circuitry 401. The debounce mechanism is further described in FIG. 8. The delay in setting the corresponding one or more touch buttons cause a delay in illuminating next one or more light emitting elements of the set of light emitting elements 301. When the user 103 steps on the illuminated one or more light emitting elements on the exercising mat 105, corresponding one or more touch buttons coupled with the illuminated one or more light emitting elements detect movement of the user 103 performing the sequence of exercise routine. The detected movement is shared to the application interface 109 from the electronic circuitry 401. To that end, the controller 501 checks for a new data corresponding to the detected movement at step 623. In some cases, the one or more touch buttons may not detect the movement. For instance, the user 103 may skip to step on the one or more the touch buttons, or pause for some time, while performing the predetermined sequence of exercise routine. In such cases, there may be no detection of the movement and the controller 501 may not have a data to share to the application interface 109. If there is no new data to send to the application interface 109, the controller 501 proceeds to step 629. In case, the controller 501 detects the new data corresponding to the detected movement, then at step 625, the controller 501 checks if one or more touch buttons that detected the movement of the user 103 are correct. In some example embodiments, the controller 501 may check if the one or more touch buttons that detected the movement are present in an array representation of required touch buttons that are required to be stepped on for performing the predetermined sequence of exercise routine. The array representation of required touch buttons may correspond to the light sequence that guides the user 103 for performing the predetermined sequence of exercise routine on the exercising mat 105. If the one or more touch buttons that detected the movement are present in the array representation of required touch buttons, then at step 627, the controller 501 shares data corresponding to the one or more touch buttons that detected the movement to the application interface 109. If the one or more touch buttons that detected the movement are not present in the array representation of required touch buttons, the process moves to step 629.

At step 629, the controller 501 is again configured to move to a sleep state, also referred to as controller 501 sleeps for a predefined time-period, such as 10 ms. After the sleep time-period expires, the controller 501 loops back to the step 605 and the steps 607-629 are repeated.

In some embodiments, the exercising mat 105 needs to be initialized before performing the steps 607-629 discussed in conjunction with the method 600 of FIGS. 6A — 6B. The exercising mat 105 may be initialized by debugging the electronic circuitry 401, which is described next in

FIG. 7 shows a flowchart illustrating a process 700 for initializing the electronic circuitry 401 of the exercising mat 105, in accordance with one or more example embodiments. The process 700 is performed by the controller 501. The process 700 begins at step 701. At step 703, a serial port for debugging the electronic circuitry 401 is initialized. At step 705, input/output ports of the electronic circuitry 401 are initialized. At step 707, access points on the electronic circuitry 401 are started using network credentials, such as a connection identifier and a password of the network 111. The connection identifier corresponds to a network identifier such as a service set identifier (SSID) of the network 111. At step 709, a wireless connection address, such as an Internet Protocol (IP) address of the wireless connection is printed to a serial port on the electronic circuitry 401. At step 711, a state value, and a current state of each of the set of touch buttons is set to a false value and “NONE”, respectively.

At step 713, control for each of the set of light emitting elements is turned off. At step 715, the system 101 hosting the application interface 109 is started. In some embodiments, the system 101 corresponds to a web socket server that allows communication with the application interface 109. The process 700 ends at step 717.

In some embodiments, the controller 501 is also configured for activating a debounce mechanism to delay in illuminating next one or more light emitting elements of the set of light emitting elements based on a determined state of each of the set of touch buttons. The state of the touch buttons may be of different types, which is described next in FIG. 8.

FIG. 8 illustrates different states 800 in a debounce mechanism corresponding to activating and displaying of the predetermined sequence of exercise routine, in accordance with one or more example embodiments. The different states 800 include an initial state 801, a triggered state 805, a send-1 state 807, a sent-on state 809 and a send-0 state 811. At the initial state 801, a state value of each of the set of touch buttons may correspond to a state value 803. For example, the state value 803 may be “NONE”. The state value may correspond to one of a true value or a false value depending on flow of current on the corresponding touch button of the set of touch buttons. The state value changes to the true value when the user 103 steps on a touch button of the set of touch buttons for performing the predetermined sequence of exercise routine. When the state value is a true value, the initial state 801 changes to a triggered state 805. The triggered state 805 corresponds to triggering a debounce wait time when the state value corresponds to the true value. When the debounce wait time is triggered, the debounce mechanism is activated. The activation of the debounce mechanism triggers the delay in setting the corresponding one or more touch buttons and in illuminating next one or more light emitting elements based on the triggered debounce wait time.

In the triggered state 805, the controller 501 may send a request to the electronic circuitry 401 to delay in triggering subsequent touch button of the set of touch buttons for a debounce time. The debounce time corresponds to a time-period to introduce the delay in triggering the subsequent touch button. The delay in triggering the subsequent touch button prevents random closures of one or more touch buttons that may cause multiple signals and signal spike through ground. The touch button is in the triggered state 805 until the closure of touch button is released. For instance, the closure of the touch button may be released when the user 103 steps away from the corresponding touch button, while performing a movement of the predetermined sequence of exercise routine. When the closure of the touch button is released, the state value changes to the false value and the triggered state 805 returns to the initial state 801. To that end, the debounce wait time is cancelled when the state value corresponds to the false value.

Further, when the debounce wait is activated, the triggered state 805 is changed to the send-1 state 807. In the send-1 state 807, the touch button is set to send a signal corresponding to the processed button. After the button is processed, the button remains in the sent-on state 809. In the sent-on state 809, the button waits for expiration of the debounce time. When the debounce time expires, the sent-on state 809 changes to the send-0 state 811. At the send-0 state 811, the debounce time expires. After the expiration of the debounce time, the send-0 state returns to the initial state 801 with the value 803.

Thus, the debounce mechanism delays in setting the subsequent one or more touch buttons and the subsequent illumination of one or more light emitting elements on the exercising mat 105, which in turn avoids changes in operation of the electronic circuitry 401.

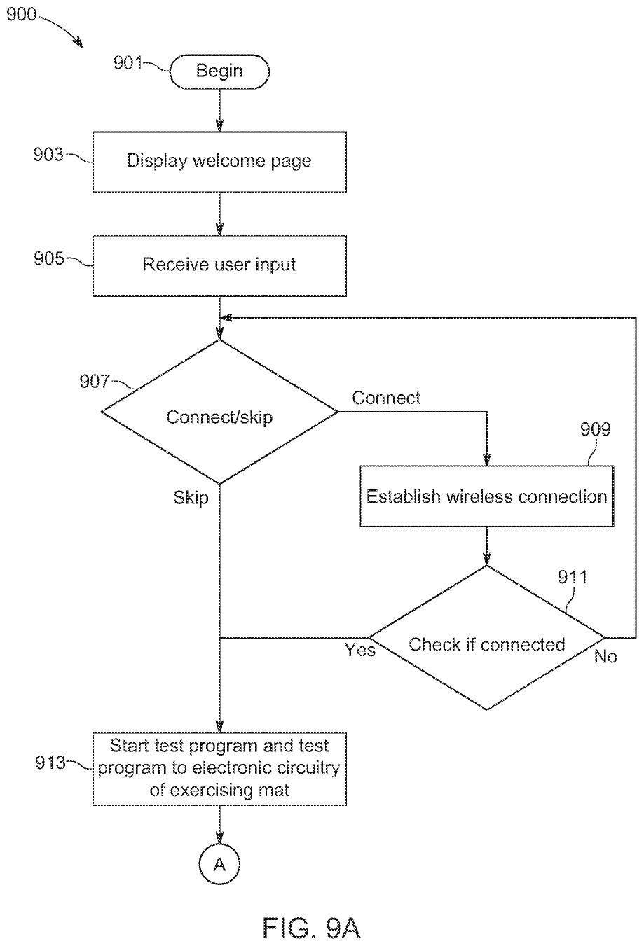

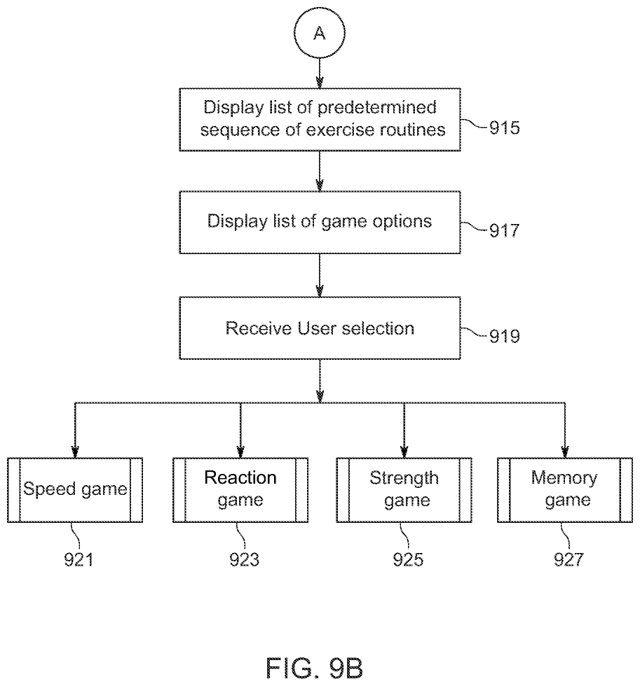

In some embodiments, the application interface 109 displays a list of predetermined sequence of exercise routines and a list of game options that include one or more of a mental and physical coordination game option, a memory game option, a physical strength game option and a speed movement game option. The user 103 may select a desired option from the list of predetermined sequence of exercise routines and the list of game options for performing movements occurring in one or more anatomical planes on the exercising mat 105. The steps for testing and selection of the desired option for performing the movements are described next in FIGS. 9A and 9B.

FIGS. 9A and 9B shows a flowchart illustrating a process 900 corresponding to the application interface 109, in accordance with one or more example embodiments. The process 900 begins at step 901. The user 103 may click on the application interface 109 in the user device 107. At step 903, the application interface 109 displays a welcome page. At step 905, the application interface 109 requests for a user input, such as an IP address from the user 103 for connecting with the electronic circuitry 401 of the exercising mat 105 An example corresponding to the welcome page and the user input for the IP address is illustrated in the GUI 201 FIG. 2. After the IP address is inputted, the application interface 109 may receive another user input that may correspond to checking connectivity with the electronic circuitry 401. In some cases, the user 103 may prefer to skip the step of checking the connectivity with the electronic circuitry 401. In such cases, the user input may correspond to skipping the step of checking the connectivity. To that end, at step 907, the application interface 109 checks if the user input corresponds to checking the connectivity with the electronic circuitry 401 or skipping the step of checking the connectivity. If the application interface 109 receives the user input that corresponds to check the connectivity, then at step 909, the application interface 109 establishes a wireless connection (e.g., a Wi-Fi connection) using the IP address received at the step 905. In one example embodiment, the electronic circuitry 401 connects to the application interface 109 using the Wi-Fi module 507 (as mentioned earlier in description of FIG. 5). If the application interface 109 receives the user input that corresponds to skipping the step of checking the connectivity, then the process moves to step 913.

After the establishment of the wireless connection, at step 911, the application interface 109 checks connectivity with the electronic circuitry 401. If there is no connectivity with the electronic circuitry 401, the process loops back to step 909. If there is connectivity with the electronic circuitry 401, go to step 913. At step 913, the application interface 109 starts a test program, such as an LED test program to test the set of light emitting elements 301. The application interface 109 sends the test program to the electronic circuitry 401. The electronic circuitry 402 receives the test program and executes the test program to test the set of light emitting elements 301 on the exercising mat 105. Upon execution of the test program, each light emitting element of the set of light emitting elements 301 is emitted.

After completing the test of the set of light emitting elements 301, the application interface 109 displays a list of predetermined sequence of exercise routines to the user 103, at step 915. The displayed list of predetermined sequence of exercise routines is shown in GUI 205 of FIG. 2. The user 103 selects a predetermined sequence of exercise routine (e.g., a lunge exercise) from the list of predetermined sequence of exercise routines. After the selection of the predetermined sequence of exercise routine, at step 917, the application interface 109 displays a list of game options to the user 103. The list of game options is shown in GUI 207 of FIG. 2. The list of game options may include a speed game option, a reaction game option, a strength game option, and a memory game option.

At step 919, the application interface 109 receives a user selection for a game option from the list of game options. If the user selection corresponds to the speed game option, then at step 921, a speed game session is initiated. If the user selection corresponds to the reaction game option, then at step 923, a reaction game session is initiated. If the user selection corresponds to the strength game option, then at step 925, a strength game session is initiated. If the user selection corresponds to the memory game option, then at step 927, a memory game session is initiated.

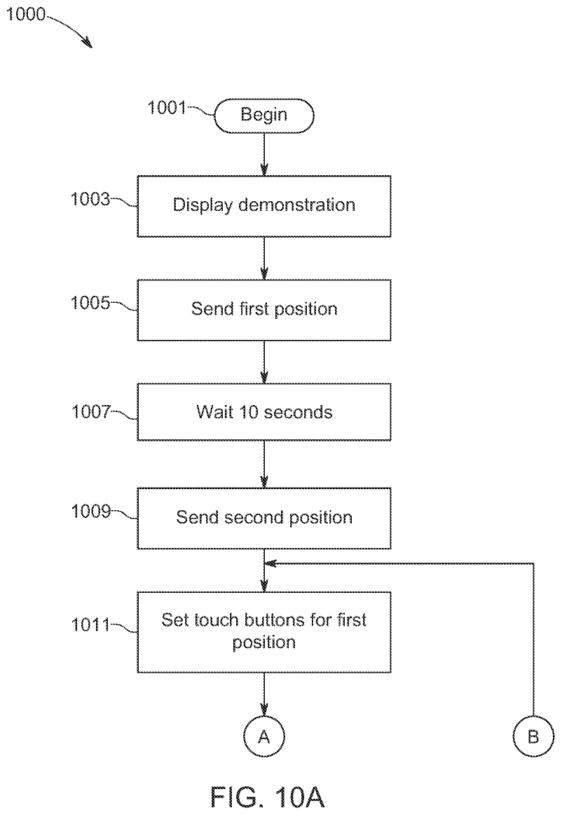

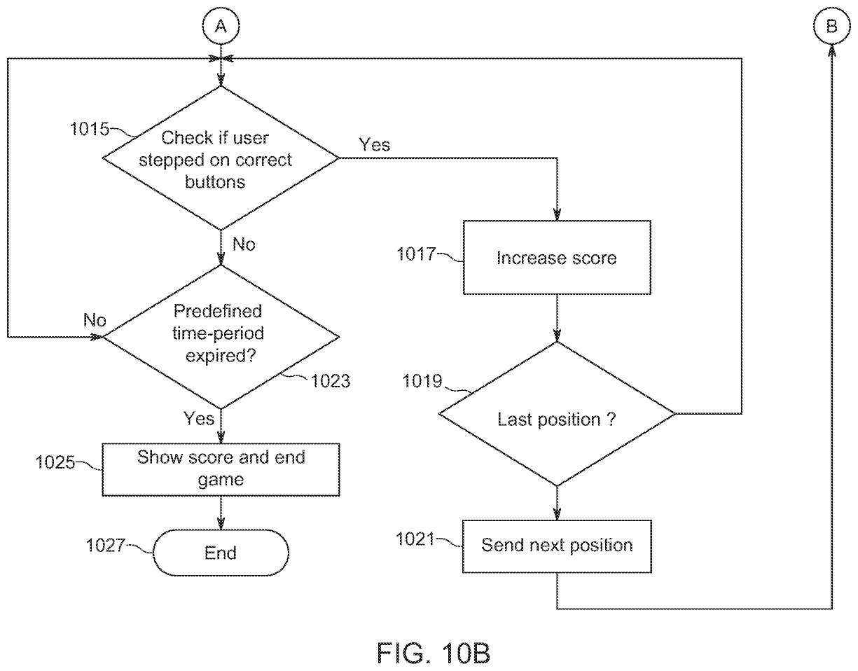

The application interface 109 may display a demonstration for the selected game option, such as a speed game corresponding to the selected predetermined sequence of exercise routine, e.g., lunge exercise. The user 103 may learn on how to play the speed game for the lunge exercise from a demonstration in the application interface 109. In order to guide the user 103 for performing the predetermined sequence of exercise routine on the exercising mat 105, the application interface 109 shares a light sequence of one or more light emitting elements corresponding to the predetermined sequence of exercise routine. In some cases, the user 103 may select a speed game option for the predetermined sequence of exercise routine, which is described in FIGS. 10A and 10B.

FIGS. 10A and 10B shows a flowchart illustrating a process 1000 corresponding to a selected predetermined sequence of exercise routine for a speed game option, in accordance with one or more example embodiments.

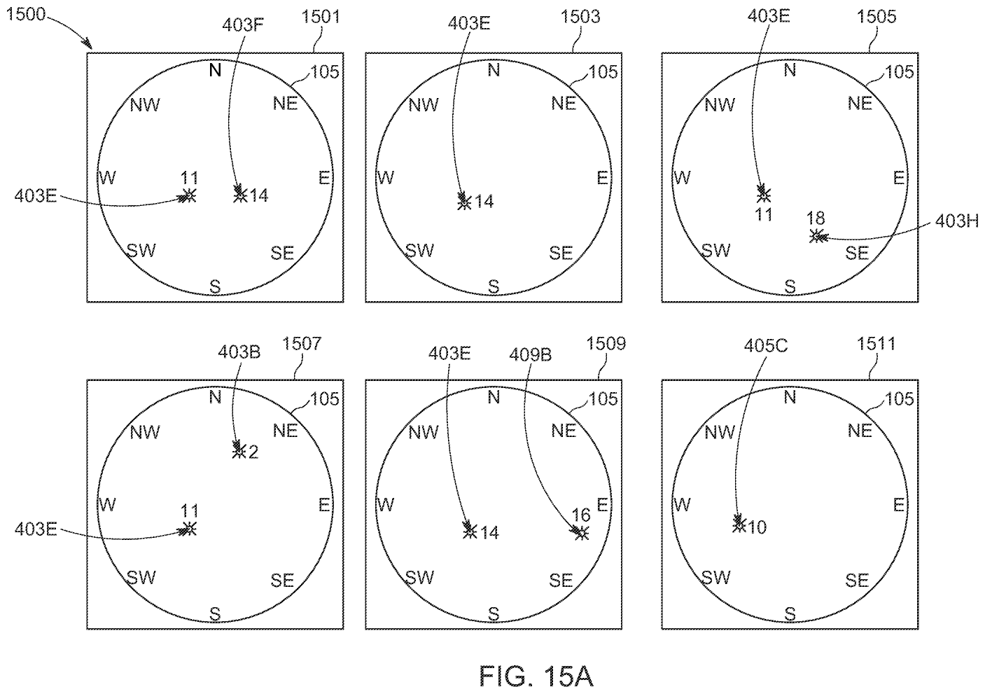

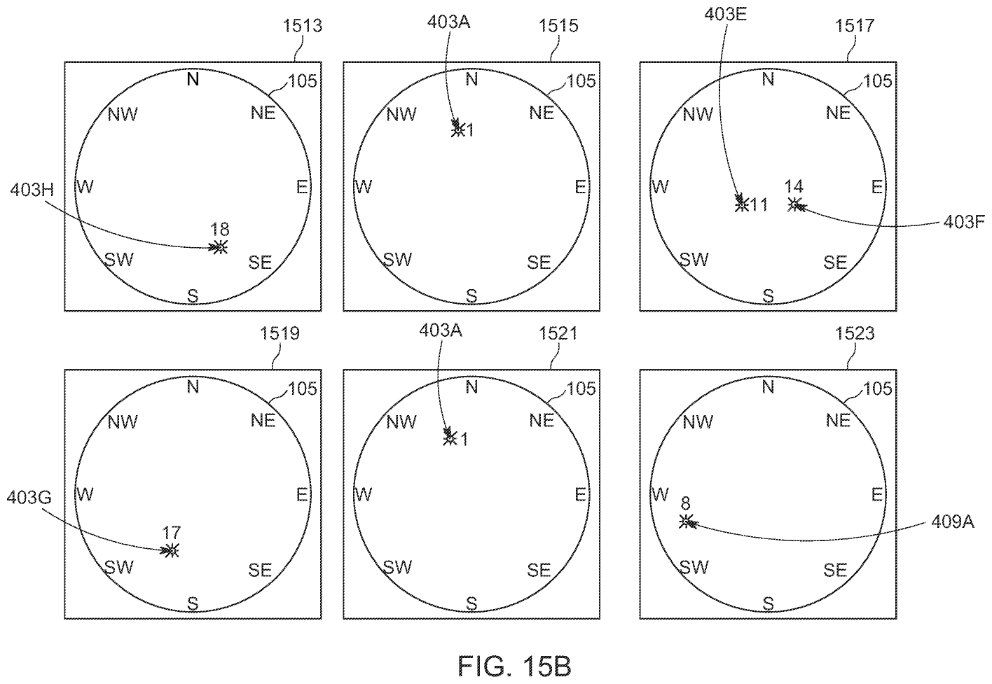

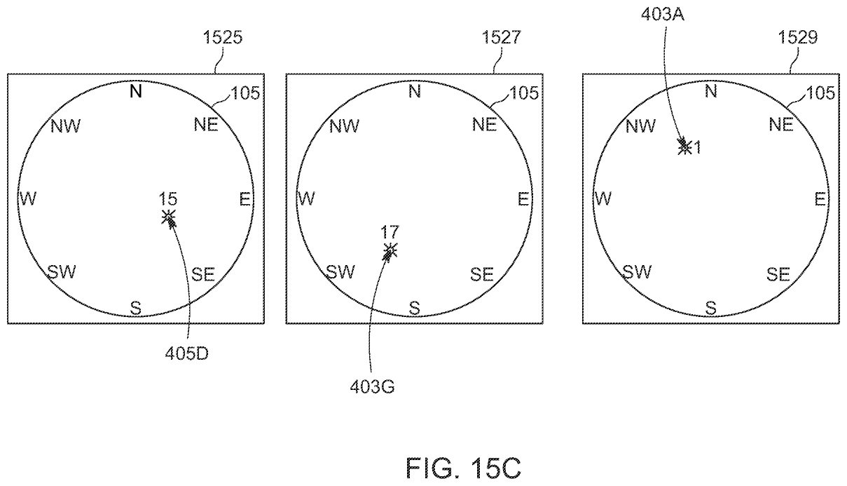

At step 1001, the process 1000 begins. At step 1003, a demonstration on how to play a selected predetermined sequence of exercise routine for a speed game option is displayed in the application interface 109. Upon completion of the demonstration, the application interface 109 may share a light sequence to the electronic circuitry 401 of the exercising mat 105. The light sequence includes one or more light emitting elements of the set of light emitting elements 310 to be illuminated on the exercising mat 105 for guiding the user 103 for performing the predetermined sequence of exercise routine on the exercising mat 105. To that end, at step 1005, the application interface 109 sends a request to trigger one or more light emitting elements of the set of light emitting elements 301 to the electronic circuitry 401 for guiding a first position of the predetermined sequence of exercise routine on the exercising mat 105. The start position is guided by the illuminating one or more light emitting elements on the exercising mat 105. The illumination of the one or more light emitting elements guiding the first position will be described in FIG. 15A. In an example embodiment, the one or two light emitting elements may be synchronized with corresponding one or two virtual light emitting elements of a set of virtual light emitting elements of the application interface 109. The corresponding one or two virtual light emitting elements may display a visual representation of these illuminating one or two light emitting elements in the application interface 109.

When the one or more light emitting elements guiding the first position are triggered, corresponding one or more touch buttons are activated, as described earlier in FIG. 6. The activated one or more touch buttons detect a movement when the user 103 steps on the illuminated one or more light emitting elements. The activation of the one or more touch buttons is delayed by the debounce mechanism to prevent changes in subsequent illumination of one or more light emitting elements corresponding to next second position. The debounce mechanism triggers a debounce wait time, such as a time-period of 10 seconds to delay the trigger. The debounce wait time may be shared to the application interface 109 as the application interface 109 is synchronized to the exercising mat 105 via the electronic circuitry 401. To that end, at step 1007, the application interface 109 waits for a predefined wait time-period, such as 10 seconds before triggering next one or more light emitting elements on the exercising mat 105. After the pre-defined wait time-period is completed, the application interface 109 triggers the next one or more light emitting elements indicative of a second position for the predetermined sequence of exercise routine, at step 1009.

At step 1011, actual one or more light emitting elements of the set of light emitting elements 301 are set to guide the first position to the user 103 based on the triggered one or more virtual light emitting elements. At step 1015, the application interface 109 checks if the user 103 pressed on the correct one or more light emitting elements on the exercising mat 105. At step 1017, the application interface 109 increases a score for the user 103 when the one or more touch buttons on the exercising mat 105 corresponding to the triggered one or more light emitting elements are correctly pressed by the user 103. After increasing the score, the next one or more light emitting elements for the next position are triggered. At step 1019, the application interface 109 checks if a position is last position of the predetermined sequence of exercise routine. At step 1021, the application interface 109 triggers another virtual one or more light emitting elements of the set of light emitting elements 301 indicative of another position of the predetermined sequence of exercise routine.

At step 1023, the application interface 109 checks if a predefined time-period, such as 10 seconds has expired. At step 1025, the application interface 109 displays total score for the user 103 for performing the predetermined sequence of exercise routine and ends session for performing the predetermined sequence of exercise routine. At step 1027, the process 1000 ends.

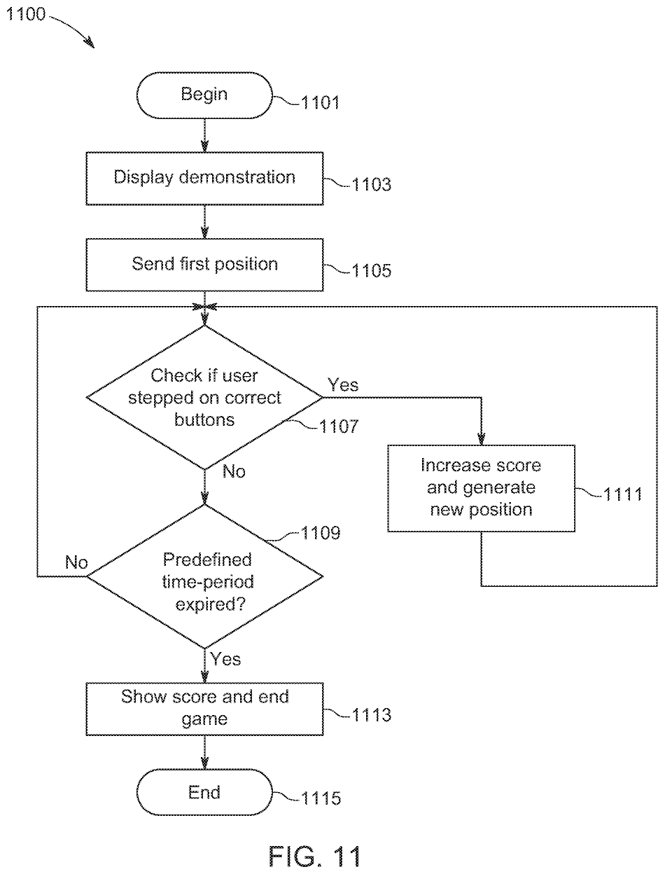

In some cases, the user 103 may select a reaction game option for the predetermined sequence of exercise routine, which is described in FIG. 11.

FIG. 11 shows a flowchart illustrating a process 1100 for performing corresponding to a selected predetermined sequence of exercise of a selected game option, in accordance with one or more example embodiments. The selected game option corresponds to a reaction game option. At step 1101, the process 1100 begins.

At step 1103, a demonstration on how to perform a selected predetermined sequence of exercise routine for a reaction game option is displayed in the application interface 109. In an example embodiment, the application interface 109 sends a light sequence corresponding to the predetermined sequence of exercise routine. The light sequence include one or more light emitting elements of the set of light emitting elements to be triggered on the exercising mat 105. At step 1105, one or more light emitting elements of the set of light emitting elements 301 in the light sequence, indicative of a first position for the selected predetermined sequence of exercise routine are triggered from the application interface 109 to the electronic circuitry 401 of the exercising mat 105. The triggered one or more light emitting elements indicative of the first position are illuminated. The user 103 steps on the illuminated one or more light emitting elements and corresponding one or more touch buttons connected to the illuminated one or more light emitting elements detect movement of the user 103. The electronic circuitry 401 sends information corresponding to the one or more touch buttons that detected the movement to the application interface 109. This indicates the user 103 correctly performed the indicated first position.

To that end, at step 1107, the application interface 109 checks if information corresponding to the one or more touch buttons that detected the movement is received from the electronic circuitry. If the application interface 109 does not receive the information corresponding one or more touch buttons that detected the movement, then the process moves to step 1109.

If the information corresponding to the one or more touch buttons that detected the movement is received from the electronic circuitry 401, then at step 1111, the application interface 109 increases a score for the user 103 for the memory game. and generates a new position for the user 103. The new position may be indicated by subsequent one or more light emitting elements. The subsequent one or more light emitting elements is triggered after a debounce mechanism is activated, as described earlier. After the new position is generated, the process moves to step 1109. At step 1109, the application interface 109 waits for a predefined time-period, such as 10 seconds.

After the predefined time-period has expired, at step 1113, the application interface 109 displays the score for the user 103 for performing the predetermined sequence of exercise routine and ends the reaction game session. At step 1115, the process 1100 ends.

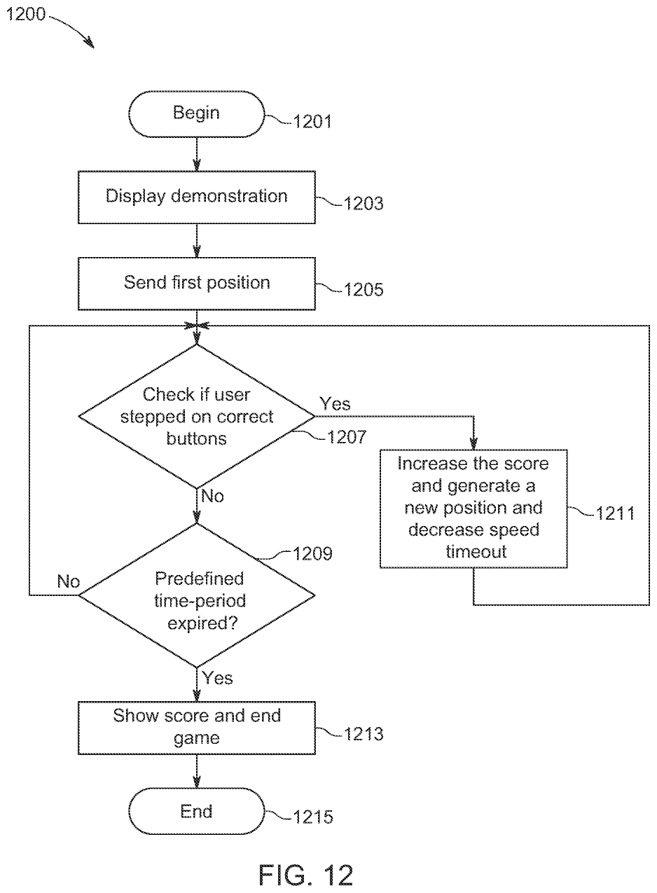

In some cases, the user 103 may select a strength game option for the predetermined sequence of exercise routine, which is described in FIG. 12.

FIG. 12 shows a flowchart illustrating a process 1200 corresponding to a selected predetermined sequence of exercise of a selected game option, in accordance with one or more example embodiments. The selected game option corresponds to a strength game option.

At step 1201, the process 1200 begins. At step 1203, a demonstration on how to perform the selected predetermined sequence of exercise routine for the strength game option is displayed in the application interface 109. After the demonstration, the application interface 109 sends a light sequence corresponding to the predetermined sequence of exercise routine for the strength game option. The light sequence includes one or more light emitting elements of the set of light emitting elements to be triggered on the exercising mat 105. At step 1205, the application interface 109 sends a request to trigger one or more light emitting elements indicative of a first position of the predetermined sequence of exercise routine. The one or more light emitting elements indicative of the first position are illuminated and corresponding one or more touch buttons are activated based on the trigger. When the user 103 steps on the illuminated one or more light emitting elements, corresponding one or more touch buttons detect the movement of the user 103. The electronic circuitry 401 sends information corresponding one or more touch buttons that detected the movement to the application interface 109.

At step 1207, the application interface 109 checks information corresponding to the one or more touch buttons that detected the movement is received from the electronic circuitry 401. The information corresponding one or more touch buttons that detected the movement indicates that the user 103 has correctly performed the corresponding position of the predetermined sequence of exercise routine. If the application interface 109 does not receive the information corresponding one or more touch buttons that detected the movement, then at step 1209, the application interface 109 checks if a predefined time-period, such as 10 seconds has expired.

If the information corresponding to the one or more touch buttons that detected the movement is received from the electronic circuitry 401, then at step 1211, the application interface 109 increases a score for the user 103. The application interface 109 also generates a new position for the user 103 and decreases speed timeout for the user 103.

At step 1213, the application interface 109 displays score for the user 103 for performing the predetermined sequence of exercise routine and ends the strength game session for the predetermined sequence of exercise routine. At step 1215, the process 1200 ends.

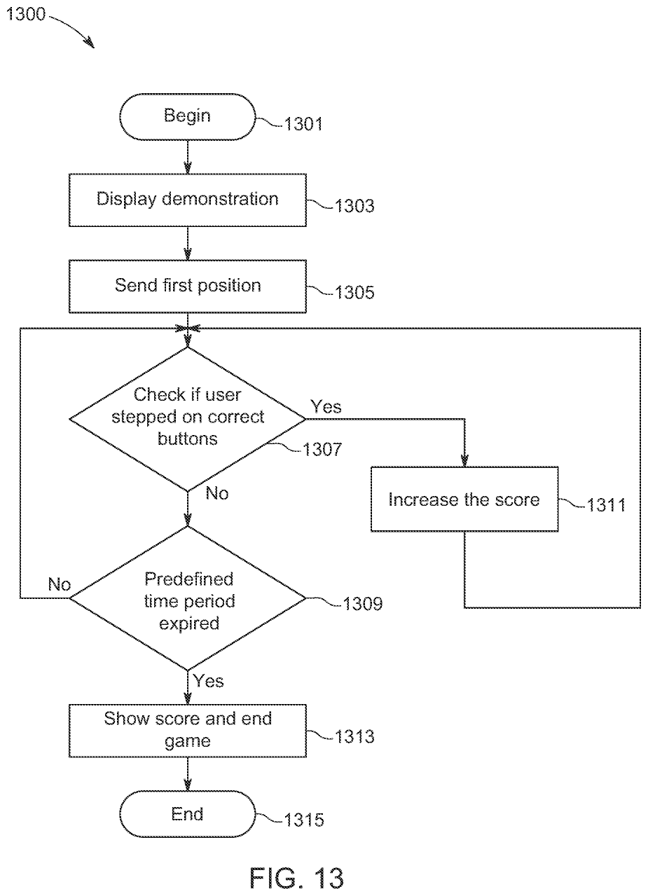

In some cases, the user 103 may select a memory game option for the predetermined sequence of exercise routine, which is described in FIG. 13.

FIG. 13 shows a flowchart illustrating a process 1300 corresponding to a selected predetermined sequence of exercise of a selected option game option, in accordance with one or more example embodiments. The selected game option corresponds to a memory game option.

At step 1301, the process 1300 begins. At step 1303, a demonstration on how to perform the selected predetermined sequence of exercise routine for the strength game option is displayed in the application interface 109. After the demonstration, the application interface 109 sends a light sequence corresponding to the predetermined sequence of exercise routine for the strength game option. The light sequence includes one or more light emitting elements of the set of light emitting elements to be triggered on the exercising mat 105. To that end, at step 1305, the application interface 109 sends a request to trigger one or more light emitting elements indicative of a first position of the predetermined sequence of exercise routine to the electronic circuitry 401. The one or more light emitting elements indicative of the first position are illuminated and corresponding one or more touch buttons are activated based on the trigger. When the user 103 steps on the illuminated one or more light emitting elements, corresponding one or more touch buttons detect the movement of the user 103. The electronic circuitry 401 sends information corresponding one or more touch buttons that detected the movement to the application interface 109. The application interface 109 checks at step 1307, if information corresponding to the one or more touch buttons that detected the movement is received from the electronic circuitry 401. The information corresponding one or more touch buttons that detected the movement indicates that the user 103 has correctly performed the corresponding position of the predetermined sequence of exercise routine. If the application interface 109 does not receive the information corresponding one or more touch buttons that detected the movement, then at step 1309, the application interface 109 waits for a predefined time-period, such as 10 seconds.

If the information corresponding to the one or more touch buttons that detected the movement is received from the electronic circuitry 401, then at step 1311, the application interface 109 increases a score for the user 103 for the memory game. After the score is increased, the process moves to step 1309. After the predefined time-period has expired, the application interface 109 shows the score to the user 103 and ends the memory game at step 1313.

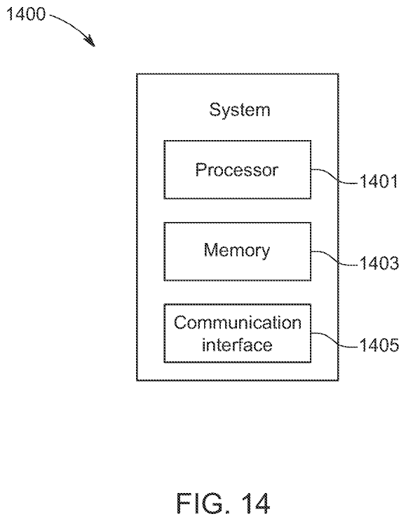

FIG. 14 illustrates a block diagram of a system 1400 hosting the application interface 109 for displaying the predetermined sequence of exercise routine, in accordance with one or more example embodiments. The system 1400 corresponds to the system 101 of FIG. 1. The system 1400 includes a processing means such as at least one processor 1401 and a storage means such as a memory 1403. The system 1400 further includes a communication interface 1405 which is configured to communicate with the user device 107 via the application interface 109.

The processor 1401 may be embodied in several different ways. For example, the processor 1401 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 1401 may include one or more processing cores configured to perform independently. A multi-core processor may enable multiprocessing within a single physical package. Additionally, or alternatively, the processor 1401 may include one or more processors configured in tandem via a bus to enable independent execution of instructions, pipelining and/or multithreading.

Additionally, or alternatively, the processor 1401 may include one or more processors capable of processing large volumes of workloads and operations to provide support for big data analysis. In an example embodiment, the processor 1401 may be in communication with the memory 1403 via a bus. The memory 1403 may be non-transitory and may include, for example, one or more volatile and/or non-volatile memories. In other words, for example, the memory 1403 may be an electronic storage device (for example, a computer readable storage medium) comprising gates configured to store data (for example, bits) that may be retrievable by a machine (for example, a computing device like the processor 1401). The memory 1403 may be configured to store information, data, content, applications, instructions, or the like, for enabling the system 1400 to carry out various functions in accordance with an example embodiment of the present invention. Additionally, the memory 1403 comprises one or more data modules that may be configured as user database 113 for storing the digital identities, the public keys, and the like. Further, the memory 1403 may be configured to buffer input data for processing by the processor 1401. As exemplarily illustrated in FIG. 4, the memory 1403 may be configured to store computer-executable instructions for execution by the processor 1401. As such, whether configured by hardware or software methods, or by a combination thereof, the processor 1401 may represent an entity (for example, physically embodied in circuitry) capable of performing operations according to an embodiment of the present invention while configured accordingly. Thus, for example, when the processor 1401 is embodied as an ASIC, FPGA or the like, the processor 1401 may be specifically configured hardware for conducting the operations described herein. Alternatively, as another example, when the processor 1401 is embodied as an executor of software instructions, the instructions may specifically configure the processor 1401 to perform the algorithms and/or operations described herein when the instructions are executed. However, in some cases, the processor 1401 may be a processor specific device (for example, a fixed computing device) configured to employ an embodiment of the present invention by further configuration of the processor 1401 by instructions for performing the algorithms and/or operations described herein. The processor 1401 may include, among other things, a clock, an arithmetic logic unit (ALU) and logic gates configured to support operation of the processor 1401.

FIGS. 15A, 15B and 15C illustrates an example use case 1500 for displaying a predetermined sequence of exercise routine, in accordance with one or more example embodiments. The predetermined sequence of exercise routine corresponds to a lunge routine for a reaction game option. The lunge routine for the reaction game option is guided by a light sequence on the exercising mat 105. The light sequence guides a series of positions 1501-1529 corresponding to the lunge routine.

As shown in FIG. 15A, a start position, such as position 1501 is indicated by light emitting elements at the point 403E and the point 403F. The light emitting elements at the point 403E and the point 403F guide the user 103 to place left foot on the point 403E and to place right foot on the point 403F. When the user 103 places each of the left foot and right foot on the corresponding points 403E and 403F, touch buttons at each of the points 403E and 403F are switched on. These movements are detected by the touch buttons and signaled to the application interface 109 by the electronic circuitry 401 of the exercising mat 105. In particular, the electronic circuitry 401 sends information corresponding to the touch buttons that detected the movement to the application interface 109. The information indicates the user 103 has correctly stepped on the illuminated light emitting elements at the points 403E and 403F. When the movement is detected, state of each of the touch buttons at the points 403E and 403F is determined (described in FIG. 8). A debounce mechanism is activated based on the determined state so as to delay in setting the next touch buttons and thereby delay in illuminating next one or more light emitting elements on the exercising mat 105. The debounce mechanism is activated for a debounce wait time corresponding to the delay in illuminating the next one or more light emitting elements. For instance, when the user 103 moves from the start position 1501 to next position, such as position 1503, there is a delay in illuminating light emitting elements at point 403.

Further, the application interface 109 starts generating scores for the user 103 from the start position 1501 based on the movements detected by one or more touch buttons, such as the touch buttons at the points 403E and 403F. Likewise, the application interface 109 increments the scores for the user 103 when consequent positions, such as positions 1503-1529, are correctly performed by the user 103.

In next position 1503, the light emitting element at the point 403E remains glowing indicating the user 103 to keep placing the left foot. The light emitting element at the point 403F turns off indicating the user 103 to lift the right foot. At position 1505, light emitting element at point 403H glows indicating the user 103 to shift the right foot on the point 403H. The shift from the position 1503 to the position 1505 indicates lunging back with the right foot.

At position 1507, the light emitting element at the point 403H turns off when the user 103 steps on the point 403H and light emitting element at point 403B glows. The light emitting element at point 403B indicates the user 103 to lunge forward with the right foot by stepping on the point 403B. The movements at the positions 1503, the position 1505 and the position 1507 include backward movement and forward movement that are in the sagittal plane of movement.

Further, in position 1509, the light emitting element at the point 403B turns off and light emitting element at point 409B glows. At position 1511, light emitting element at point 405C glows when the right foot steps on the point 409B. The position 1509 guided by the light emitting elements at the points 403E and 409B indicates a lateral lunge with the right foot. The position 1511 guided by the light emitting element at point 405C indicates an opposite side lateral lunge with the right foot. The lateral lunges performed in such positions 1509 and 1511 allow the user 103 to move in the frontal plane of movement.

At position 1513, light emitting element at point 403H glows and the light emitting element at the point 405C turns off, when the right foot is placed on the point 405C. The light emitting element at the point 403H guides the user 103 to externally rotate with the right foot. At position 1515, light emitting element at point 403A glows when the right foot steps on the point 403H. The light emitting element at the point 403A guides the user 103 to perform an opposite side internal rotation lunge with the right foot. The movements at the positions 1513 and the position 1515 include rotation movements which are performed in the transverse plane of movement. Further, the user 103 repeats the movements from the positions 1501 to 1513 for a number of rounds, such as 5 times. After completion of the repeated movements, all light emitting elements on the exercising mat 105 glow and an audio output is generated via the speaker 513 of the exercising mat 105. The audio output may include an instruction change legs for next upcoming positions, such as positons 1517-1529.

At position 1517, the light emitting elements at the points 403E and 403F glow guiding the user 103 to return to a start position, such as the position 1501. At position 1519, light emitting element at point 403G glows when the left footsteps on the point 403E. The right foot remains on the point 403F. The light emitting element at the point 403G guides the user 103 to place the left foot on the point 403G for performing a posterior back lunge. At position 1521, the light emitting element at the point 403A glows guiding the user 103 to step the left foot on the point 403G for performing a forward lunge. The posterior back lunge and the forward lunge movements allow the user 103 to move in the sagittal plane.

At position 1523, a light emitting element at point 409A glows when the left foot steps on the point 403A, which guides the user 103 to perform a side lateral lunge by stepping the left foot on the point 409A. At position 1525, a light emitting element at point 405D glows when the left footsteps on the point 409A. The light emitting element at the point 405D guides the user 103 to perform an opposite side lateral lunge. The lateral lunge movements in the positions 1523 and 1525 allow the user 103 to move in the frontal plane.

At position 1527, a light emitting element at point 403G glows when the left footsteps on the point 405D. The light emitting element at the point 403G guides the user 103 to perform an external rotation lunge. At position 1529, the light emitting element at the point 403A glows when the left footsteps on the point 403G. The light emitting element at the point 403A guides the user 103 to perform an external rotation lunge in an opposite side. The rotation lunge movements in the positions 1527 and 1529 allow the user 103 to move in the transverse plane.

The repeated movements are performed in the different planes of movement, such as the sagittal, the frontal and the transverse planes, which mentally and physically involves the user 103 in performing the lunge routine. The mental and physical involvement enables improvement of mental and physical coordination, while achieving an optimal physical strength.

After completion of the lunge routine for the reaction game option, the application interface 109 displays a time duration taken to complete the routine and a number of rounds of the routine performed by the user 103. The application interface 109 also displays a statistical report indicative of a progress rating of the user 103 for performing the routine of the particular game option, i.e., the reaction game option.

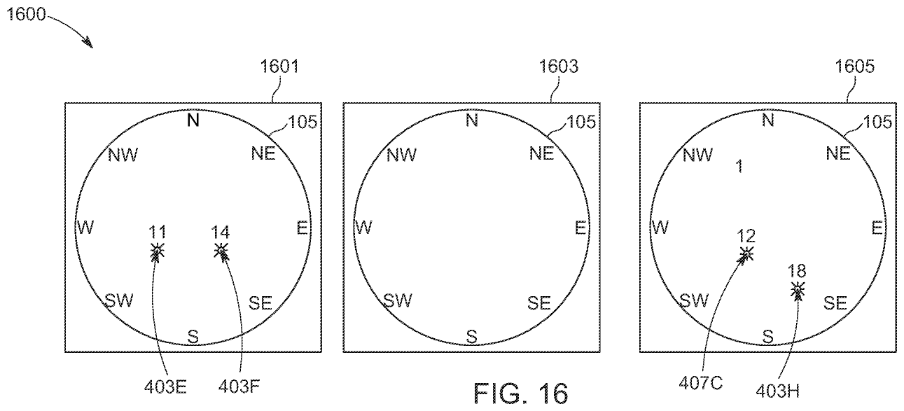

FIG. 16 illustrates another example an example use case 1600 for displaying a predetermined sequence of exercise routine, in accordance with one or more example embodiments. The predetermined sequence of exercise routine corresponds to a lunge routine for a memory game option. The lunge routine for the memory game option is guided by a light sequence on the exercising mat 105. The light sequence guides a series of positions 1601-1605 corresponding to the lunge routine. In the memory game for the lunge routine, the user 103 is trained to memorize sequence of one or more light emitting elements displayed in random on the exercising mat 105. The randomized sequence of one or more light emitting elements is derived from a light sequence for guiding the lunge routine.

At a start position 1601, light emitting elements at points 403E and 403F glow guiding the user 103 to place the left foot and right foot on each corresponding points on the exercising mat 105. For instance, the left foot is placed on the point 403E and the right foot is placed on the point 403F. After the user 103 steps on the points 403E and 403F, the light emitting elements are turned off. As shown in FIG. 16, at position 1603, no light emitting elements glow indicating the user 103 to memorize previous position, such as the position 1601.

Further, corresponding touch buttons at the point 403E and 403F are switched on (not shown) upon turning off the light emitting elements at the points 403E and 403F. When the touch buttons at the point 403E and 403F are switched on, a light emitting element at point 407C and a light emitting element at point 403H are turned on. The light emitting elements at the points 407C and 403H are turned off after glowing for a predefined time-period, such as 10 seconds.

In this manner, consequent positions of the lunge routine are guided by the randomize sequence of turning on and off the one or more light emitting elements. This helps the user 103 to memorize the movements for the lunge routine. When the user 103 steps correctly, scores are incremented and displayed in the application interface 109 upon completion of the lunge routine for the memory training. For instance, the user 103 gives an input to complete the training. The application interface 109 displays a time duration taken to complete the lunge routine and a number of rounds of the lunge routine performed by the user 103. The application interface 109 also displays a statistical report indicative of a progress rating of the user 103 for performing the lunge routine.

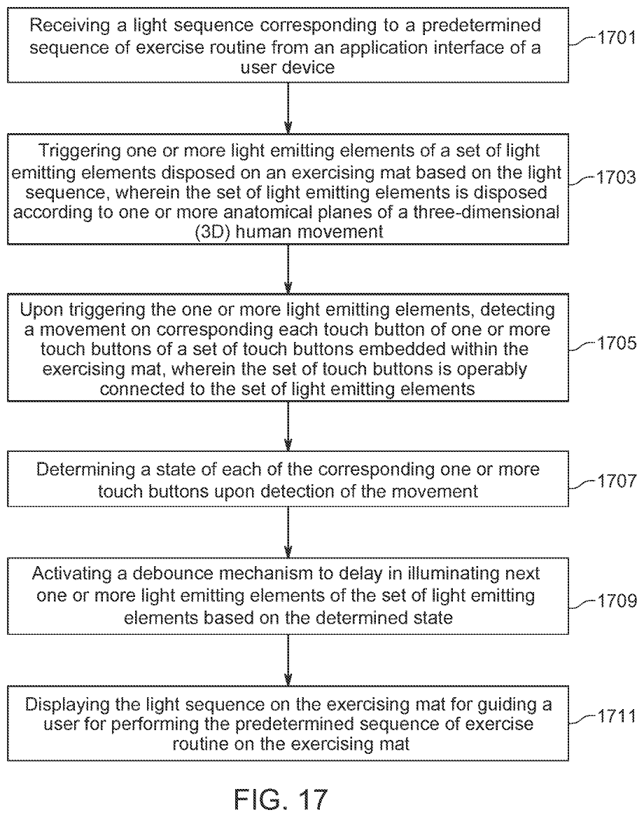

FIG. 17 shows a method 1700 for displaying the predetermined sequence of exercise routine, in accordance with one or more example embodiments. The method 1700 is executed by the electronic circuitry 401 of the exercising mat 105. Starting at step 1701, the method 1700 includes receiving a light sequence corresponding to the predetermined sequence of exercise routine from an application interface (e.g., the application interface 109) of a user device (e.g., the user device 107). In an example embodiment, the application interface 109 receives a user selection for the predetermined sequence of exercise routine from a list of predetermined sequence of exercise routines and a user selection for a game option from a list of game options (as shown in GUI 205 and GUI 207 of FIG. 2 and FIGS. 9A and 9B).

At step 1703, the method 1700 includes triggering one or more light emitting elements of a set of light emitting elements disposed on the exercising mat 105 based on the light sequence (as shown in FIGS. 15A, 15B and 15C and FIGS. 16). The set of light emitting elements is disposed according to one or more anatomical planes of a three-dimensional (3D) human movement.

At step 1705, the method 1700 includes upon triggering the one or more light emitting elements, detecting a movement on corresponding each touch button of corresponding one or more touch buttons of a set of touch buttons on the exercising mat. The set of touch buttons is operably connected to the set of light emitting elements.

At step 1707, the method 1700 includes determining a state of each of the corresponding one or more touch buttons upon detection of the movement.

At step 1709, the method 1700 includes activating a debounce mechanism to delay in illuminating next one or more light emitting elements of the set of light emitting elements based on the determined state (as shown in FIG. 8).

At step 1711, the method 1700 includes displaying the light sequence on the exercising mat for guiding a user for performing the predetermined sequence of exercise routine on the exercising mat.

In this manner, the display of the light sequence guides the user to perform the predetermined sequence of exercise routine in the anatomical planes, and thereby may improve physical and mental health of the user. Further, the exercising mat arranged with the light emitting elements that display the light sequence and the corresponding touch buttons to detect the user movement, are triggered with a delay corresponding to a debounce mechanism, which avoids any faulty in operation of the circuitry of the exercising mat. The debounce mechanism may improve feasibility and efficiency for displaying the predetermined sequence of exercise routine. Furthermore, the game options may engage the user to perform the predetermined sequence of exercise routine with ease.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

We claim:

1. A computer-implemented method for performing a predetermined exercise routine on an exercising mat, comprising:
receiving a light sequence corresponding to the predetermined exercise routine from a user device;
triggering one or more light emitting elements of a set of light emitting elements disposed on the exercising mat based on the received light sequence, wherein the set of light emitting elements is disposed according to one or more anatomical planes of a three-dimensional (3D) human movement and wherein the one or more light emitting elements are associated with movements corresponding to the predetermined exercise routine;
upon triggering the one or more light emitting elements, detecting a movement on corresponding each touch button of one or more touch buttons on the exercising mat, wherein the one or more touch buttons is operably connected to the one or more light emitting elements;
determining a state of each of the corresponding one or more touch buttons upon detection of the movement; and
displaying the light sequence on the exercising mat for guiding a user for performing the predetermined exercise routine on the exercising mat.

2. The method of claim 1, further comprising a debounce mechanism to delay illumination of a light emitting element of the one or more light emitting elements based on the determined state.

3. The method of claim 2, wherein the debounce mechanism is configured to perform at least:
determining a state value of the determined state, wherein the state value corresponds to one of a true value and a false value;
triggering a debounce wait time when the state value corresponds to the true value; and
activating the delay based on the debounce wait time.

4. The method of claim 3, wherein further comprising:
cancelling the debounce wait time when the state value corresponds to the false value; and
deactivating the delay based on the cancellation of the debounce wait time.

5. The method of claim 1, wherein receiving the light sequence further comprises:
receiving a wireless connection address from an application interface of the user device;
establishing a wireless connection to the application interface based on the wireless connection address;
upon establishment of the wireless connection, synchronizing the set of light emitting elements and the one or more touch buttons with a corresponding set of virtual light emitting elements and a set of virtual touch buttons in the application interface; and
receiving the light sequence for the predetermined exercise routine.

6. The method of claim 5, further comprising:
receiving a test program from the application interface;
executing a test of the set of light emitting elements based on the test program; and
illuminating each light emitting element of the set of light emitting elements based the execution of the test program.

7. The method of claim 1, further comprising:
displaying, on the user device, a list of predetermined exercise routines; and
receiving, at the user device, a user selection corresponding to the predetermined exercise routine from the list of predetermined exercise routines.

8. The method of claim 7, further comprising:
displaying, on the user device, a list of training options for the selected predetermined exercise routine, wherein the list of training options comprises one or more of a mental and physical coordination training option, a memory training option, a physical strength training option and a speed movement training option; and
receiving, at the user device, a user selection of one of a training option from the list of training options.

9. The method of claim 8, further comprising:
receiving, at the user device, a user input for marking a completion of the selected predetermined exercise routine; and
displaying, on the user device, a time duration for the completion of the predetermined exercise routine and a number of rounds performed by the user corresponding to the completion of the predetermined exercise routine.

10. The method of claim 9, further comprising:
displaying, on the user device, a statistical report indicative of a progress rating of the user corresponding to the completion of the predetermined exercise routine.

11. A system for displaying a predetermined exercise routine, comprising:
an application interface on a user device;
an exercising mat, wherein the exercising mat comprises:
a set of light emitting elements disposed on a top surface of the exercising mat according to one or more anatomical planes of a three-dimensional (3D) human movement,
a set of touch buttons operably coupled with the set of light emitting elements, and
an electronic circuitry integrated with the exercising mat, wherein the electronic circuitry is configured to:
receive a light sequence corresponding to the predetermined exercise routine from the application interface;
trigger one or more light emitting elements of the set of light emitting elements based on the light sequence;
upon triggering the one or more light emitting elements, detect a movement on each touch button of corresponding one or more touch buttons of the set of touch buttons;
determine a state of each of the corresponding one or more touch buttons upon detection of the movement; and
display the light sequence on the exercising mat for guiding a user for performing the predetermined exercise routine.

12. The system of claim 11, further comprising a debounce mechanism to delay illumination of one or more light emitting elements of the set of light emitting elements based on the determined state.

13. The system of claim 12, wherein for the activation of the debounce mechanism, the electronic circuitry is further configured to:
determine a state value of the determined state, wherein the state value corresponds to one of a true value and a false value;
trigger a debounce wait time when the state value corresponds to the true value; and
activate the delay based on the debounce wait time.

14. The system of claim 13, wherein the electronic circuitry is further configured to:
cancel the debounce wait time when the state value corresponds to the false value; and
deactivate the delay based on the cancellation of the debounce wait time.

15. The system of claim 11, wherein for receiving the light sequence, the electronic circuitry is further configured to:
receive a wireless connection address from the application interface;
establish a wireless connection to the application interface based on the wireless connection address;
upon establishment of the wireless connection, synchronize the set of light emitting elements and the set of touch buttons with a corresponding set of virtual light emitting elements and a set of virtual touch buttons in the application interface; and
receive the light sequence associated with the predetermined exercise routine.

16. The system of claim 15, wherein the electronic circuitry is further configured to:
receive a test program from the application interface;
execute a test of the set of light emitting elements based on the test program; and
emit each light emitting element of the set of light emitting elements based on the execution of the test program.

17. The system of claim 11, wherein the application interface is further configured to:
display a list of predetermined exercise routines; and
receive a user selection corresponding to the predetermined exercise routine from the list of predetermined sequence of exercise routines.

18. The system of claim 17, wherein the application interface is further configured to:
display a list of training options for the selected predetermined exercise routines, wherein the list of training options comprises one or more of a mental and physical coordination training option, a memory training option, a physical strength training option and a speed movement training option; and
receive a user selection of one of a training option from the list of training options.

19. The system of claim 18, wherein the application interface is further configured to:
receive a user input for indicating a completion of the selected predetermined exercise routine; and
display a time duration for the completion of the predetermined exercise routine and a number of rounds performed by the user corresponding to the completion of the predetermined exercise routine.

20. A computer program product comprising a non-transitory computer readable medium having stored thereon computer executable instruction which when executed by at least one processor, cause the at least one processor to carry out operations for displaying a predetermined exercise routine on an exercising mat, the operations comprising:
receiving a light sequence corresponding to the predetermined exercise routine from an application interface of a user device;
triggering one or more light emitting elements of a set of light emitting elements disposed on the exercising mat based on the light sequence, wherein the set of light emitting elements is disposed according to one or more anatomical planes of a three-dimensional (3D) human movement;
upon triggering the one or more light emitting elements, detecting a movement on corresponding one or more touch buttons of a set of touch buttons on the exercising mat, wherein the set of touch buttons is operably connected to the set of light emitting elements;
determining a state of each of the corresponding one or more touch buttons upon detection of the movement; and
displaying the light sequence on the exercising mat for guiding a user for performing the predetermined exercise routine.
